(19) United States
(12) Patent Application Publication
(10) Pub. No.: US 2001/0006793 A1
(43) Pub. Date: Jul. 5, 2001

(54) MODULATORS OF EUKARYOTIC CASPASES

(76) Inventors: MARY-ANN BJORNSTI, PHILADELPHIA, PA (US); DAVID HALL, PHILADELPHIA, PA (US); JASON KANG, DREXEL HILL, PA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(21) Appl. No.: 09/045,620

(22) Filed: Mar. 20, 1998

(51) Int. Cl. C12Q 1/37; C12Q 1/02; G01N 33/53; C12N 15/09; A61K 38/00

(52) U.S. Cl. 435/23; 435/24; 435/29; 435/7.8; 435/69.2; 514/21

(57) ABSTRACT

The invention includes numerous caspase inhibitors and methods of identifying and using them. The invention also includes a yeast cell comprising an isolated nucleic acid encoding a caspase and a gene vector encoding a caspase.

### FIELD OF THE INVENTION

The field of the invention is modulation of apoptosis.

### BACKGROUND OF THE INVENTION

Numerous diseases of humans involve inappropriate levels of apoptosis in one or more tissues, and are herein designated “misapoptotic diseases.” Misapoptotic diseases include those characterized by too little apoptosis, such as cancers, viral infections, and various autoimmune disorders, and those characterized by too much apoptosis, such as AIDS, neurodegenerative disorders (e.g. Alzheimer's disease or Parkinson's disease), ischemic injuries, myelodysplastic syndrome, and toxin-induced liver diseases.

Apoptosis is a process of programmed cell death, whereby a eukaryotic cell, in response to specific stimuli, initiates a series of physiological events which ultimately lead to cell death. In higher eukaryotes, apoptosis is associated with the processing and activation of aspartic acid specific cysteine proteases known as caspases. Expression of a functional caspase requires expression of catalytically inactive zymogen precursors of caspase subunits and proteolytic cleavage of the zymogen precursors to yield catalytically active caspase subunits which constitute a functional caspase. The functional caspase generated by this process can then exert its proteolytic activity on cellular proteins (Takahashi et al., 1996, Proc. Natl. Acad. Sci. USA 93:8395-8400; Kumar, 1995, Trends Biol. Sci. 20:198-202; Femandez-Alnemri et al., 1994, J. Biol. Chem. 269:30761-30764). Known eukaryotic caspases include, but are not limited to, caspases 1-10 (Cohen, 1997, Biochem. J. 326:1-16). Another aspartic acid specific protease which has been associated with apoptosis is granzyme B.

Two viral proteins, designated p35 and CrmA, have been identified which are capable of inhibiting caspase activity (Villa et al., 1997, Trends Biol. Sci. 22:-388-393. X-linked IAP is also an inhibitor of caspase activity (Deveroux et al., 1997, Nature 388:300-303).

Modulators of caspase activity can be used for treatment of misapoptotic diseases in humans (Fernandez-Alnemri et al., 1994, J. Biol. Chem. 269:30761-30764; Haecker et al., 1994, Trends Biol. Sci. 19:99-100; Fisher, 1994, Cell 78:539-542; Thompson, 1995, Science 267:1456-1462; Bump et al. 1995, Science 269:1885-1888). Significant, unmet needs remain for caspase modulators and for methods of identifying them. Caspase modulators could be used to treat misapoptotic diseases in humans such as neurodegenerative disorders, cancers, stroke, myocardial infarction, and AIDS, among others. The present invention satisfies these needs.

### BRIEF SUMMARY OF THE INVENTION

The invention relates to a method of determining whether a test compound is a caspase modulator. The method comprises providing a functional eukaryotic caspase to a first yeast cell, providing the test compound to the first cell, and comparing a viability indicator of the first cell with the same viability indicator of a second yeast cell which was provided the caspase and which was not provided the test compound, wherein a difference between the viability indicator of the first cell and the viability indicator of the second cell is an indication that the compound is a caspase modulator. The first cell and the second cell may be of the same species, and may, for example, be selected from the group consisting of a Saccharomyces cerevisiae cell and a Schizosaccharomyces pombe cell. The Saccharomyces cerevisiae may, for example, be selected from the group consisting of strain FY250 and strain MBY5.

In one embodiment of this method of the invention, the caspase is provided to the interior of the first cell by providing a caspase vector to the first cell. The caspase vector comprises an isolated nucleic acid encoding a self-activating caspase and a promoter operably linked to the isolated nucleic acid. In one aspect, the caspase vector further comprises a second isolated nucleic acid encoding a self-activating form of a second eukaryotic caspase. Both of the isolated nucleic acids may, for example, be operably linked to the same promoter. The promoter may, for example, be selected from the group consisting of an inducible promoter, a repressible promoter, and a promoter which is both inducible and repressible. In an aspect of this embodiment, the promoter is an inducible promoter such as the GAL1 promoter, and the first cell is incubated first in the absence of an inducer of the promoter and thereafter in the presence of the inducer.

In another embodiment of this method of the invention, the caspase is provided to the first cell by providing an expressible vector encoding the caspase to the first cell and thereafter expressing the caspase.

In yet another embodiment of this method of the invention, the caspase is provided to the first cell by integrating an isolated nucleic acid encoding the caspase into the genome of the first cell and thereafter expressing the caspase.

The caspase used in this method of the invention may, for example, be selected from the group consisting of human caspase 1, human caspase 2, human caspase 3, human caspase 4, human caspase 5, human caspase 6, human caspase 7, human caspase 8, human caspase 9, human caspase 10, and granzyme B.

The viability indicator used in this method of the invention may, for example, be selected from the group consisting of cell number, cell refractility, cell fragility, cell size, number of cellular vacuoles, a stain which distinguishes live cells from dead cells, methylene blue stain, bud size, bud location, nuclear morphology, and nuclear staining.

The invention also relates to an anti-caspase gene vector. This gene vector comprises an isolated nucleic acid which encodes a caspase inhibitor selected from the group consisting of human cytochrome b, human tat binding protein, human mitochondrial loop attachment site, a glutamate-binding subunit of a human NMDA receptor complex, human myelin basic protein, human synaptophysin p38, human snRNP protein B, human protein 1, human ubiquitin C-terminal hydrolase, human tissue inhibitor of metalloprotease-3, human MHC HLA-DRw12-MHC class II β chain, human transglutaminase, human death associated protein 1, and human hnRNP D.

The invention further relates to a method of modulating apoptosis in a eukaryotic cell. This method of the invention comprises providing a caspase modulator to the interior of the cell, wherein the caspase modulator is selected from the group consisting of human cytochrome b, human tat binding protein, human mitochondrial loop attachment site, a glutamate-binding subunit of a human NMDA receptor complex, human myelin basic protein, human synaptophysin p38, human snRNP protein B, human protein 1, human ubiquitin C-terminal hydrolase, human tissue inhibitor of metalloprotease-3, human MHC HLA-DRw12-MHC class II β chain, human transglutaminase, human death associated protein 1, and human hnRNP D. In one embodiment of this method of the invention, the caspase modulator is a caspase inhibitor, and the cell is an affected cell in a human patient afflicted with a misapoptotic disease. In another embodiment of this method of the invention, the caspase modulator is a caspase activator, and the cell is an affected cell in a human patient afflicted with a misapoptotic disease.

The invention still further relates to a yeast cell comprising an isolated nucleic acid which encodes a caspase, the isolated nucleic acid being operably linked to a promoter.

In addition, the invention relates to gene vector comprising an isolated nucleic acid encoding a caspase, the isolated nucleic acid being operably linked to an inducible promoter. In one embodiment of this gene vector of the invention, the gene vector further comprises a second isolated nucleic acid encoding a second caspase. The second isolated nucleic acid is also operably linked to the same promoter.

### BRIEF DESCRIPTION OF THE DRAWINGS

FIG. 1 is a graph which depicts the viability of FY250 yeast cells which were transformed with vectors described herein in Example 1. “Vectors” cells were transformed with vector alone. “Caspase 8β” cells were transformed with a vector encoding the self-activating caspase-8β, under transcriptional control of the GAL1 promoter.

FIG. 2 is a graph which depicts the viability of FY250 yeast cells which were transformed with vectors described herein in Example 1. “Vectors” cells were transformed with vector alone. “Caspase-3” cells were transformed with a vector encoding (non-self-activating) caspase-3, under transcriptional control of the GAL1 promoter. “Caspase-8β/-3” cells were transformed with a vector encoding the self-activating caspase-8β, under transcriptional control of the GAL1 promoter and with a vector encoding caspase-3, also under transcriptional control of the GAL1 promoter.

### DETAILED DESCRIPTION

The invention relates to the discovery that capsases expressed by higher eukaryotes, such as humans, can be expressed in cells of lower eukaryotes, such as yeast cells, which do not normally express caspases. When such yeast cells express caspases of higher eukaryotes and the caspases are activated by cleavage of the zymogen precursors, cytotoxicity results. Caspase-induced cytotoxicity in yeast cells is phenotypically similar to caspase-associated apoptosis in cells of higher eukaryotes. Thus, yeast cells which expresses a functional caspase can be used as a model of apoptosis. Such models are useful for identifying modulators of caspases, for identifying compositions and methods useful for treating misapoptotic diseases, and for other purposes.

Yeast cells, such as Saccharomyces cerevisiae cells or Schizosaccharomyces pombe cells, can be genetically manipulated more simply than can cells of higher eukaryotes. In addition, methods for culturing yeast cells are well established. Furthermore, the absence of naturally-occurring caspases in yeast cells permits investigation of the properties of an individual caspase enzyme expressed in such yeast cells without interference from other caspase enzymes which might be present in a higher eukaryote which naturally expresses the individual caspase enzyme. Thus, yeast cells provide an advantageous model system for identifying modulators of caspases.

The invention relates to compositions and methods for identifying modulators of eukaryotic caspases, and further relates to compounds which have been identified as caspase modulators and to methods of using those compounds.

Definitions

As used herein, the following terms have the meanings herein associated with them.

The term “caspase” means an aspartic acid specific cysteine protease associated with apoptosis in a eukaryotic cell. It is understood that caspases are expressed in an inactive zymogen form, and that activation of the zymogen subunits, effected by proteolytic cleavage of those subunits, is necessary to yield mature, enzymatically active caspase. It is understood that isoforms of individual caspases exist, each isoform being encoded by the same gene. By way of example, α and β isoforms of caspase 8 have been identified, and are both encoded by the same gene. Discussion herein of a caspase is intended to refer to all isoforms of the caspase. For example, discussion of caspase 8 refers to the α and β isoforms of caspase 8. It is recognized that granzyme B, an aspartic acid specific protease associated with apoptosis, has lower homology with human caspases than the human caspases have with one another. Nonetheless, the term “caspase,” as used herein, includes granzyme B.

A caspase is “functional” if the caspase exhibits the cysteine protease activity associated with a mature caspase.

It is understood that certain caspases are, as described herein, capable of catalyzing their own activation following expression. Such a caspase is referred to herein as “self-activating.”

A “caspase modulator” means a compound which increases or decreases the activity of a caspase in the presence of the compound, relative to the activity of the caspase in the absence of the compound. Caspase modulators include caspase activators, wherein the activity of a caspase is higher in the presence of the caspase activator than in its absence, and caspase inhibitors, wherein the activity of a caspase is lower in the presence of the caspase inhibitor than in its absence.

A “viability indicator” is a characteristic of a eukaryotic cell which indicates the viability of the cell. “Viability” of a eukaryotic cell refers to the ability of a eukaryotic cell to continue to live, grow, proliferate, replicate, differentiate, or develop. It is understood that numerous characteristics of a cell may be used to ascertain or to estimate the viability of a cell including, but not limited to, the viability indicators described herein. A first cell is said to be more viable than a second cell if the first cell appears to be more likely to continue to live, grow, proliferate, or develop than the second cell.

A “lower” eukaryote means a eukaryote which naturally occurs in a unicellular form. Lower eukaryotes include, but are not limited to, yeasts such as Saccharomyces cerevisiae and Schizosaccharomyces pombe.

A “higher” eukaryote means a eukaryote which naturally occurs as a multicellular organism. Higher eukaryotes include, but are not limited to, organisms such as humans, cells of which naturally express at least one caspase.

A “misapoptotic” disease of a human is a disease state characterized by an inappropriate level or rate of apoptosis among a population of cells in the human. Misapoptotic diseases include those in which excessive apoptosis occurs, such as, by way of example, neurodegenerative diseases such as Alzheimer's disease and Parkinson's disease, stroke, myocardial infarction, reperfusion injury, and AIDS. Misapoptotic disease further include those in which insufficient apoptosis occurs, such as, by way of example, cancer, a viral infection, and an autoimmune disorder.

An “affected cell” of a human afflicted with a misapoptotic disease is a cell which either would have undergone apoptosis but for the affliction of the human with the misapoptotic disease or would not have undergone apoptosis but for the affliction of the human with the misapoptotic disease.

An “isolated nucleic acid” refers to a nucleic acid segment or fragment which has been separated from the sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector; into an autonomously replicating plasmid or virus; or into the genomic DNA of a prokaryote or eukaryote; or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

By describing two isolated nucleic acids as “operably linked” is meant that a single-stranded or double-stranded nucleic acid moiety comprises each of the two isolated nucleic acids and that the two isolated nucleic acids are arranged within the nucleic acid moiety in such a manner that at least one of the two nucleic acid sequences is able to exert a physiological effect by which it is characterized upon the other.

A “vector” is a composition of matter which comprises an isolated nucleic acid and which can be used to deliver the isolated nucleic acid to the interior of a cell. Numerous vectors are known in the art including, but not limited to, linear isolated nucleic acids, linear isolated nucleic acids associated with ionic or amphiphilic compounds, plasmids, and viruses.

An “expressible vector” is a vector which comprises a nucleic acid encoding a gene product, wherein when the expressible vector is provided to a eukaryotic cell, the gene product is expressed.

A “small molecule” is a chemical compound having a size sufficiently small that it is able to diffuse across the cell membrane of a eukaryote or through a pore in the cell membrane of a eukaryotic cell.

An “inhibition vector” is a vector which comprises a caspase inhibitor or which comprises an isolated nucleic acid which encodes a caspase inhibitor, and which can be used to provide the caspase inhibitor to the interior of a cell of a eukaryote. By way of example, an inhibitor vector may be a liposome comprising a small molecule which is a caspase inhibitor or a virus vector comprising an isolated nucleic acid which encodes a polypeptide which is a caspase inhibitor.

A “gene product” means a molecule formed as a result of transcription, reverse transcription, or translation of an isolated nucleic acid comprising all or part of a gene.

Description

The invention includes a screening method of determining whether a compound is a caspase modulator. The method comprises providing a functional eukaryotic caspase to the interior of a first lower eukaryotic cell which does not normally comprise the first caspase, providing the compound to the interior of the first cell, incubating the first cell for a period of time, and comparing a viability indicator of the first cell with the same viability indicator of a second lower eukaryotic cell. The second cell does not normally comprise the first caspase, was provided the first caspase, was not provided the compound, and was incubated for approximately the same period of time as the first cell. A difference between the viability indicator of the first cell and the viability indicator of the second cell is an indication that the compound is a caspase modulator. If the viability indicator indicates that the first cell is more viable than the second cell, then the compound is a caspase inhibitor. If the viability indicator indicates that the first cell is less viable than the second cell, then the compound is a caspase activator. If the viability indicator is substantially the same for both the first and the second cell, then the compound is neither a caspase inhibitor nor a caspase activator, and is therefore not a caspase modulator.

The first and second cells may each be a Saccharomyces cerevisiae cell, such as a cell of S. cerevisiae strain FY250 or a cell of S. cerevisiae strain MBY5.

A caspase may be provided to the interior of a cell by providing a caspase vector to the cell. The caspase vector may, for example comprise an isolated nucleic acid encoding a self-activating form of the caspase and a promoter operably linked to the isolated nucleic acid. Alternately, if the cell has been provided with one or more proteolytic enzymes capable of activating the zymogen form of the caspase, then the isolated nucleic acid may encode a non-self-activating form of the caspase.

The caspase vector may further comprise a second isolated nucleic acid encoding a self-activating form of another eukaryotic caspase. The two isolated nucleic acids may be separate or they may be part of the same isolated nucleic acid. In the latter case, the isolated nucleic acids may both be operably linked to the same promoter.

The promoter of the caspase vector may be an inducible promoter, a repressible promoter, or a promoter which is both inducible and repressible.

When the promoter is an inducible promoter, the screening method of the invention may be practiced by incubating a cell in the absence of an inducer of the promoter, whereby the caspase is not expressed, and any effect of the compound on the cell may be assessed. Thereafter, the cell may be incubated in the presence of the inducer, whereby the caspase is expressed, and any effect of the compound on the activity of the caspase may be assessed by comparing a viability indicator of the cell when incubated in the presence of both the compound and the inducer with the same viability indicator when the cell is incubated in the absence of the compound and in the presence of the inducer.

When the promoter is a repressible promoter, the screening method of the invention may be practiced by incubating a cell in the presence of a repressor of the promoter, whereby the caspase is not expressed, and any effect of the compound on the cell may be assessed. Thereafter, the cell may be incubated in the absence of the repressor, whereby the caspase is expressed, and any effect of the compound on the activity of the caspase may be assessed by comparing a viability indicator of the cell when incubated in the presence of the compound and in the absence of the repressor with the same viability indicator when the cell is incubated in the absence of the compound and in the absence of the repressor.

When the promoter is one which is both inducible and repressible, such as the GAL1 promoter, the screening method of the invention may be practiced by incubating a cell in the absence of an inducer of the promoter, in the presence of a repressor of the promoter, or both, whereby the caspase is not expressed, and any effect of the compound on the cell may be assessed. Thereafter, the cell may be incubated in the presence of an inducer and in the absence of a repressor, whereby the caspase is expressed, and any effect of the compound on the activity of the caspase may be assessed by comparing a viability indicator of the cell when incubated in the presence of the compound and in the presence of the inducer, in the absence of the repressor, or both in the presence of the inducer and the absence of the repressor, with the same viability indicator when the cell is incubated in the absence of the compound and in the presence of the inducer, in the absence of the repressor, or both in the presence of the inducer and the absence of the repressor.

A caspase may be provided to a cell by providing an expressible vector encoding the caspase to the cell and thereafter expressing the caspase. Alternately, the caspase may be provided to the cell by integrating an isolated nucleic acid encoding the caspase into the genome of the cell and thereafter expressing the caspase.

The caspases described herein may be any caspase. By way of example, the caspase may be selected from the group consisting of human caspases 1-10 and granzyme B.

The viability indicator which is assessed in the screening method of the invention may be substantially any indicator of the viability of the cell. By way of example, the viability indicator may be selected from the group consisting of cell number, cell refractility, cell fragility, cell size, number of cellular vacuoles, a stain which distinguishes live cells from dead cells, methylene blue staining, bud size, bud location, nuclear morphology, and nuclear staining. Other viability indicators and combinations of the viability indicators described herein are known in the art and may be used in the screening method of the invention.

The compositions and methods described herein may be used to screen individual compounds, a plurality of compounds, or a library of compounds, for example. Because the compositions and methods described herein yield discrete results for individual compounds, those compositions and methods are amenable to repetitive, high volume use, such as is associated with high-throughput screening of compound and nucleic acid libraries.

Compounds which may be screened using the screening method of the invention include, but are not limited to, small molecules, known pharmaceutical agents, compounds which are obtained from synthetic chemical libraries, proteins, peptides, oligonucleotides, nucleic acids each encoding a gene product such as a protein, and gene products encoded by naturally-occurring organisms such as animals, plants, or viruses. The use of either single-copy or multi-copy based mammalian cDNA libraries to identify mammalian genes that suppress caspase-induced cell lethality is contemplated.

Because the caspases described herein are believed to act intracellularly, it is necessary to provide the compound to be screened for caspase-modulating activity to the interior of the cell. Any method of providing the compound to the interior of the cell may be used. In the case of small molecules and molecules capable of diffusing across the cell membrane, the compound may be provided to the interior of the cell by providing the compound to a medium which contacts the cell. Methods of using a nucleic acid vector to provide an isolated nucleic acid encoding a gene product to the interior of a cell and thereafter producing the gene product are well known. Other methods of delivering a compound to the interior of a cell include, but are not limited to, providing a composition, such as a liposome, comprising the compound and an amphiphilic agent to the first cell, bombarding the cell with a microparticle coated with the compound, and electroporating the cell in the presence of the compound.

In an alternate embodiment of the screening method of the invention, a procaspase is provided to the first cell. The compound is also provided to the first cell. A viability indicator of the first cell is assessed after the procaspase and the compound have been provided to the first cell. The same viability indicator is also assessed in a second eukaryotic cell which was provided the procaspase, but which was not provided the compound. If the first cell is less viable than the second cell, then the compound is a caspase activator.

The invention also relates to an anti-caspase gene vector comprising a nucleic acid vector and an isolated nucleic acid which encodes a caspase modulator, the modulator having been identified using the screening method of the invention as caspase modulators. Caspase modulators which have been identified include each of the compounds listed in Table 1. The nucleic acid vector used in the anti-caspase gene vector of the invention may be substantially any vector known or subsequently discovered to be useful for providing an isolated nucleic acid to the interior of a eukaryotic cell. The nucleic acid vector may, by way of example, be a DNA plasmid or a virus vector. Preferably, the nucleic acid vector is capable of delivering the isolated nucleic acid to a selected subset of cells in a mammal such as a human. Such vectors are known in the art. The anti-caspase gene vector of the invention may be used to deliver the isolated nucleic acid encoding the caspase inhibitor to the interior of a cell of a eukaryote such as a mammal, thereby inhibiting the activity of a caspase in the cell. If the eukaryote is a human patient afflicted with a misapoptotic disease characterized by too much apoptosis, then the anti-caspase gene vector of the invention may be used to prevent apoptosis of an affected cell of the patient.

TABLE 1
Caspase Modulators

| | Caspase | Caspase |
|---|---|---|
| Compound | Inhibitor | Activator |
| Human Cytochrome b | ✓ | |
| Synthetic Peptide Inhibitors z-VAD-fmk and | ✓ | |
| IETD/fmk | | |
| Human Tat Binding Protein (also known as | ✓ | |
| TBP, SUG1, and 26S proteosomal p45 | | |
| subunit ATPase) | | |
| Human Mitochondrial Loop Attachment Site | ✓ | |
| Glutamate-Binding Subunit of a Human NMDA | ✓ | |
| Receptor Complex | | |
| Human Myelin Basic Protein | ✓ | |
| Human Synaptophysin p38 | ✓ | |
| Human snRNP Protein B | ✓ | |
| Human Protein 1 (expressed from a | ✓ | |
| mitochondrial gene encoding a product designated | | |
| ‘Protein 1’) | | |
| Human Ubiquitin C-terminal Hydrolase (also | ✓ | |
| known as UHX1) | | |
| Human Tissue Inhibitor of Metalloprotease-3 | ✓ | |
| Human MHC HLA-DRw12-MHC class II β | ✓ | |
| Chain | | |
| Human Transglutaminase | ✓ | |
| Human Death Associated Protein 1 | ✓ | |
| (also known as DAP-1) | | |
| Human hnRNP D | ✓ | |
| Viral protein p35 | ✓ | |
| Caspase-8β | | ✓ |

It is understood that the screening method of the invention can be used to identify caspase modulators, and that caspase modulators identified by the screening method of the invention are modulators of apoptosis in cells which exhibit caspase-associated cell death. The screening method of the invention is thus equally considered a method of identifying caspase modulators and a method of identifying modulators of caspase-associated apoptosis.

The invention further relates to a method of modulating apoptosis in a eukaryotic cell. This method of the invention comprises providing a caspase modulator to the interior of the cell, wherein the caspase modulator is selected from the group consisting of those listed in Table 1. When the caspase modulator is a caspase inhibitor, inhibition of apoptosis in an affected cell results. Such inhibition may serve to treat a human patient afflicted with a misapoptotic disease characterized by excessive apoptosis. Such misapoptotic diseases include, by way of example, neurodegenerative diseases such as Parkinson's disease and Alzheimer's disease, stroke, myocardial infarction, reperfusion injury, and AIDS. When the caspase modulator is a caspase activator, induction of apoptosis of an affected cell results. Such induction may serve to treat a human patient afflicted with a misapoptotic disease characterized by insufficient apoptosis. Such misapoptotic disease include, by way of example, cancer, viral infections, and autoimmune disorders. Thus, the modulation method of the invention is useful for treating a human patient afflicted with a misapoptotic disease characterized by either excessive or insufficient apoptosis.

### Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Caspase-Induced Cytotoxicity in Yeast Cells

The experiments described in this Example establish that cytotoxicity may be induced in yeast cells which are transformed with a vector comprising an isolated nucleic acid comprising the self-activating caspase-8β linked to an inducible promoter and wherein the cells are thereafter contacted with an inducer of the promoter.

Yeast strain FY250 (Mat a, ura3-52, his3A200, leu2AI, trpIA63, p⁺) was used in experiments described herein. Petite strains lacking mitochondrial DNA (FY250 p⁻) were selected by incubating exponentially growing cultures of FY250 cells in synthetic complete media supplemented with 2% dextrose plus 10 μg/ml ethidium bromide for 16 hours at 30° C. The treated cells were plated onto rich media (YPD plates) supplemented with 0.1% dextrose and 3% glycerol and incubated at 30° C. After three days, petite cells (FY250p⁻) which formed smaller colonies were selected. The p⁻phenotype of the cells was confirmed by a lack of growth on YP medium containing 3% glycerol and the absence of mitochondrial DNA staining with DAPI.

cDNA encoding the self-activating caspase-8β was cloned into a yeast expression plasmid vector designated YCpGAL1-L, as described (Reid et al., J. Biol. Chem. 272:12091-12099). The cDNA which encoded caspase-8β encoded the prodomain and the p18 and p11 subunits of the caspase. The cDNA was operably linked to the galactose-inducible GAL1 promoter of an ARS/CEN vector to yield a vector designated “YCpGAL1-caspase-8β-L”. Caspase-3 cDNA was cloned into vector YCpGAL1-L to yield a vector designated “YCpGAL1-caspase-3.” A control vector, designated “YCpGAL1-L” comprised the galactose-inducible GAL1 promoter of an ARS/CEN vector, and did not encode a caspase subunit. The “L” designation indicates that each of these vectors further comprised the LEU2 gene for use as a selectable marker. The vectors YCpGAL1-L and YCpGAL1-caspase-3 each comprised the URA3 selectable marker. Thus, each of the vectors could be stably maintained by yeast cultured in synthetic complete medium lacking leucine (SC-leucine media) or in SC-leucine medium lacking uracil (SC-leucine-uracil medium).

For efficient expression in yeast, caspase-3 and caspase-8β cDNAs were cloned into the pET-21-b expression vector, and were amplified and cloned under the galactose inducible GAL1 promoter in single copy ARS/CEN vectors. These constructs encoded full length proenzymes, tagged at the N-terminus with the T7 epitope (Met—Ala—Ser—Met—Thr—Gly—Gly—Gln—Gln—Met—Gly; SEQ ID NO: 1). The C-terminus of caspase-3 contained a 6×His tag; however, the stop codon of caspase-8β preceded the 6×His tag sequence. In each case, caspase cDNA was amplified using pfu polymerase (Stratagene) and primers 5′ATGGCTAGCATGACTGGT3′ (SEQ ID NO: 2) and 5′ CCGGAATTCTCA(GTG)₆ 3′ (SEQ ID NO: 3), which correspond to the vector backbone sequences encoding the T7 and His tags, respectively. The resulting DNA fragments were phosphorylated with T4 polynucleotide kinase, purified on Qiagen spin columns and ligated into yeast expression vectors containing pGAL1.

To construct plasmid YCpGAL1-caspase-3, the URA3 marked ARS/CEN vector YCpGAL1 -TOP1 was first restricted with Bam HI and Xba I to liberate the TOP1 sequences. The ends were then repaired and ligated to the amplified DNA fragments encoding caspase-3. Plasmid YCpGAL1-caspase-8β-L was constructed by ligating the T7, His-tagged caspase cDNA into the Smal site of plasmid YCPGAL1-L (Reid et al., supra), which was derived from the ARS/CEN plasmid pRS416. The L postscript indicates the presence of the LEU2 selectable marker. For each construct, the correct orientation of the caspase cDNAs was determined by pfu polymerase amplification using primers specific for GAL1 promoter sequences and the C-terminal His tag and was confirmed by DNA sequencing.

The well-characterized yeast strain, Saccharomyces cerevisiae FY250, was transformed with YCpGAL1-L, with YCpGAL1-caspase-3, with YCpGAL1-caspase-8β-L vectors, or with both YCpGAL1-caspase-3 and YCpGAL1-caspase-8β-L vectors, to yield control transformants, caspase-3 transformants, caspase-8β transformants, and caspase-8β/−3 transformants respectively. Individual transformants or caspase-8β/−3 transformants were each selected on SC-leucine, SC-uracil, or SC-leucine-uracil medium which had been supplemented with dextrose to repress GAL1 promoter function. Individual transformants were propagated on the same selective medium comprising dextrose. Expression from the GAL1 promoter of each transformant was induced by culturing the cells first in medium comprising raffinose to alleviate repression of pGAL1, and then, at a selected initial time, pGAL1 expression was induced by addition of galactose to the medium. At selected times following the initial time, serial ten-fold dilutions of each transformant were prepared and spotted onto selective plates containing medium which comprised dextrose. The number of viable cells capable of forming colonies on these plates was subsequently determined. Cytotoxicity induced by GAL1-mediated expression of caspase-8β, caspase-3, or both, was manifested as non-viability of transformant cells when plated on selective medium.

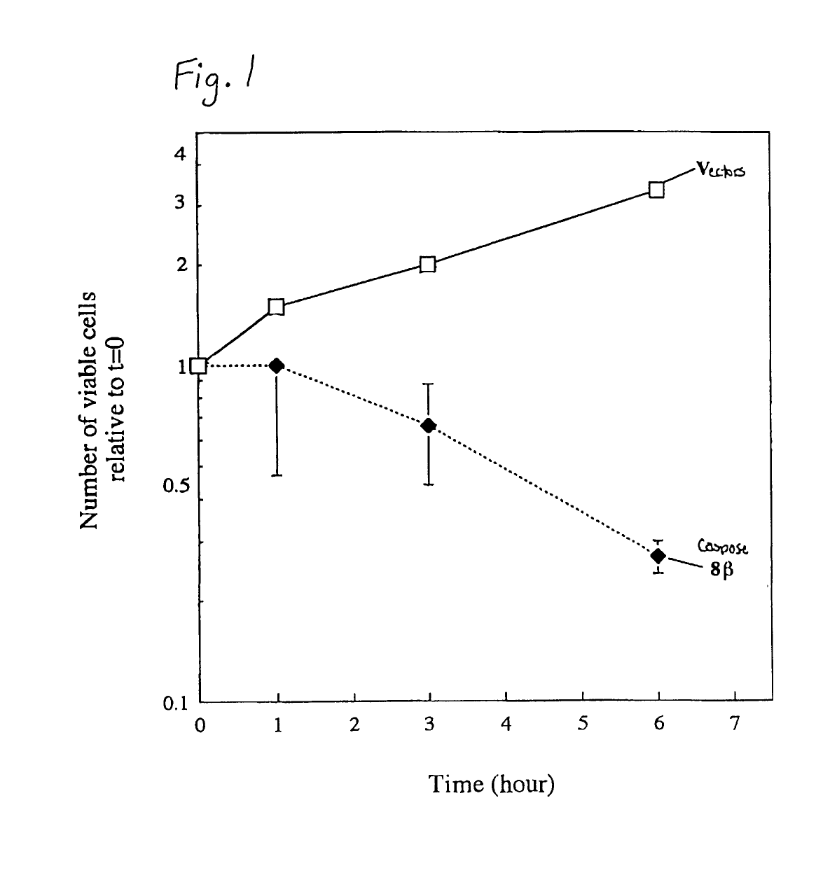

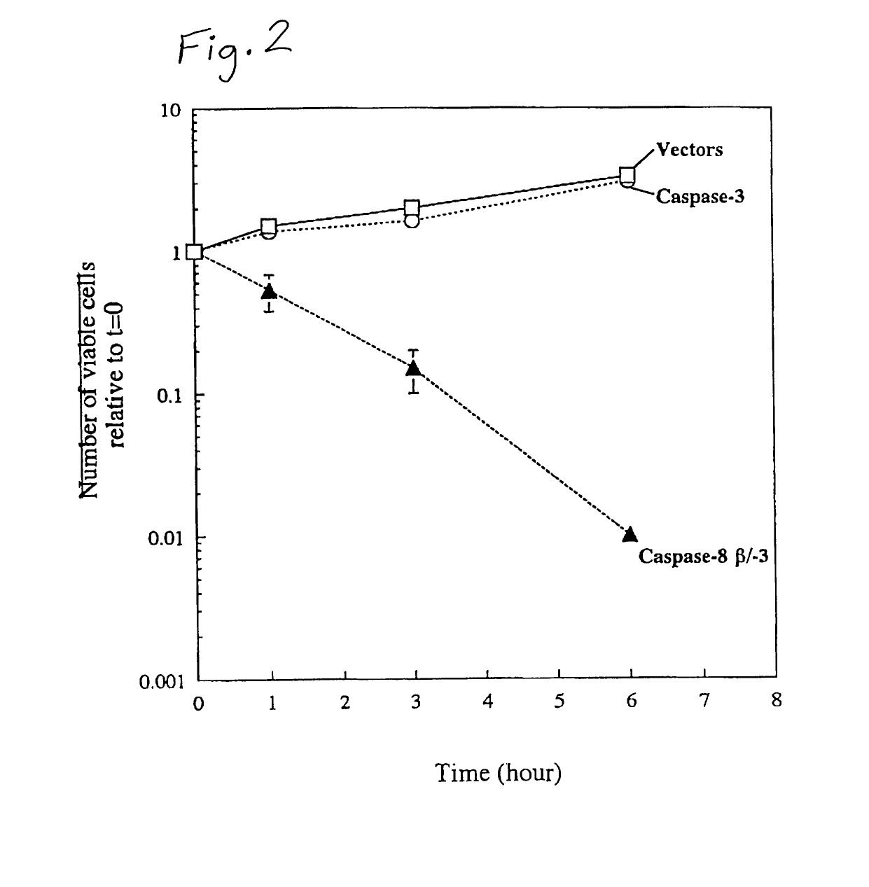

As indicated in the graphs of FIGS. 1 and 2, the viability of yeast cells transformed with YCpGAL1, with YCpGAL1-L, or with YCpGAL1-caspase-3 did not decrease following induction with galactose. However, when yeast cells which were transformed with either YCpGAL1-caspase-8β-L or with both YCpGAL1-caspase-8β-L and YCpGAL1-caspase-3 were cultured in the presence of galactose, an approximately immediate decrease in cell viability was observed. Yeast cells transformed with either YCpGAL1-caspase-8β-L or with both YCpGAL1-caspase-8β-L and YCpGAL1-caspase-3 exhibited increased cell volume, increased vacuole size, punctate nuclear staining using the DNA-specific stain designated DAPI, and DNA fragmentation. In contrast, yeast cells transformed with YCpGAL1-L or with YCpGAL1-caspase-3 did not exhibit these properties.

The characteristics of yeast cells transformed with a vector encoding self-activating caspase-8β were consistent with the characteristics of mammalian cells undergoing apoptosis. Expression of both caspase-3 and caspase-8β in yeast led to a more rapid lethal phenotype than expression of caspase-8β, as indicated in FIGS. 1 and 2. This result is also consistent with the cascade of caspase activation evident in higher eukaryotes (Cohen, 1997, Biochem. J. 326:1-16). In contrast, caspase-3 was not able to self-activate, and was not active in yeast unless acted upon by caspase-8β. Thus, the results of the experiments described in this Example indicate that S. cerevisiae strain FY250 cells transformed and induced as described in this Example may be used as a model of eukaryotic cells undergoing caspase-mediated apoptosis.

### Example 2

### A Screening Method for Identifying Caspase Inhibitors

Yeast transformants, as described in this Example, may be used to identify compounds that induce apoptosis, such as gene products, small molecules, and the like. These transformants may also be used to identify compounds that protect cells from cytotoxic effects of apoptotic agents.

A genetic screen to identify mammalian gene products, the expression of which suppresses cytotoxic action of caspases in yeast, permits identification and characterization of mammalian genes which regulate the apoptotic response induced by caspases.

Expression in S. cerevisiae of a Self-Activating Form of a Caspase A nucleic acid encoding self-activating caspase-8β was cloned into the yeast expression plasmid vector designated YCpGAL1. It is understood that any other self-activating caspase, such as caspase- 10 for example, could be used. The cDNA encoded both subunits of the caspase, and was operably linked to the galactose-inducible GAL1 promoter of an ARS/CEN vector to yield a caspase vector. The caspase vector further comprised a selectable marker (the LEU2 gene) to permit growth on selective medium. A control vector was also generated, which comprised the galactose-inducible GAL1 promoter of an ARS/CEN vector, but did not encode a self-activating caspase.

Cells in a suspension of S. cerevisiae FY250 were transformed with the caspase-8β vector to yield a caspase transformant. A separate suspension was transformed with the control vector to yield a control transformant. Individual caspase transformants were selected on SC-leucine medium supplemented with dextrose to repress GAL1 promoter function. Individual transformants were then propagated on SC-leucine medium comprising dextrose.

To assess the cytotoxic effects of caspase-8β expression, samples of caspase-8β transformants grown in SC-leucine medium comprising dextrose were obtained. Serial ten-fold dilutions of these samples were prepared and spotted onto plates containing SC-leucine medium, which comprise either dextrose or galactose. Cytotoxicity induced by GAL1-mediated expression of caspase-8β was manifested as non-viability of transformant cells when plated on selective medium or as a difference between a viability indicator of caspase-8β transformants, relative to control transformants.

Caspase-8β activity was alternately assessed by a protein cleavage assay using crude cell extracts prepared from galactose-induced cells. Crude cell extracts were prepared from cultures of yeast cells at hourly intervals following galactose induction. The presence and specific activity of caspase-8β in the cell extracts were assessed by monitoring cleavage of the fluorometric caspase-specific peptides Asp— Glu—Val—Asp—AMC (AMC is aminomethyl coumarin) and Ile—Glu—Thr—Asp—AFC (AFC is aminotrifluoromethyl coumarin). These peptides are commercially available.

The specific peptide-based assay may be performed by incubating about 50 to about 150 micrograms (circa 50 microliters) of crude cell extract with 10 microliters of a 50 micromolar solution of a fluorogenic peptide. Release of AMC is assessed spectrofluorometrically using excitation light having a wavelength of 380 nanometers and detecting emitted light having a wavelength of 460 nanometers. Release of AFC is assessed spectrofluorometrically using excitation light having a wavelength of 400 nanometers and detecting emitted light having a wavelength of 505 nanometers. In preliminary experiments, increases in the level of peptide cleavage catalyzed by extracts prepared from yeast cells expressing caspase-8β have been observed.

Results obtained using the specific peptide-based assay have been compared with cell viability data to establish a direct correlation between cell viability and caspase-8β activity. Cell viability can readily be established by observing morphological changes such as loss of refractility and the accumulation of a large number of vacuoles in caspase transformants. Staining of yeast cultures using the fluorescent FUN-1 cell stain (Molecular Probes, Inc) may be performed to distinguish live cells (which stain orange or red) from dead cells (which stain yellow or green). Positive staining using methylene blue may also be used to indicate the presence of dead cells. Cells may further be examined for the acquisition of a terminal phenotype, such as that observed in cdc mutants (Weinert et al., 1994, Genes Dev. 8:652-665; Pringle et al., 1981, In: The Molecular Biology of the Yeast Saccharomvces, Cold Spring Harbor, N.Y., pp. 97-142). In addition, other characteristics such as bud size and location or nuclear morphology may be assessed following Calcoflour and DAPI staining, respectively. Flow cytometry (as described in Kauh et al., 1995, Proc. Natl. Acad. Sci. USA 92:6299-6303), for example, may be used to conform cell cycle arrest induced by caspase activation.

Preliminary experiments indicated that the loss of refractility, positive staining with methylene blue, generation of giant cells with large vacuoles, and changes in nuclear morphology in caspase-8β transformants were consistent with caspase-8β-induced cell death, rather than a simple inhibition of cell growth.

Induction of DNA damage, induction of expression of inducible genes such as RNR3, DIN3, and SAD1 using lacZ reporter constructs may also be monitored following galactose induction of caspase expression, in order to detect caspase-mediated events.

Screening to Identify Compounds Capable of Suppressing Caspase-Induced Lethality

A yeast transformant which expresses a self-activating form of a caspase, as described in this Example, can be used to identify compounds that are capable of inhibiting caspase activity. As described herein, the transformant expresses the caspase when an inducer of the inducible promoter of the caspase vector is provided to the caspase transformant. When the caspase transformant expresses the caspase, cytotoxicity ensues in the caspase transformant. If an inhibitor of the caspase is provided to the caspase transformant prior to the onset of cytotoxicity, the caspase transformant may be enabled to survive expression of the caspase or the onset of cytotoxicity may be delayed. Preferably, the caspase inhibitor is provided to the caspase transformant prior to induction of caspase expression.

Known inhibitors of caspases include viral proteins designated p35, CrmA, and X-IAP (Bump et al. 1995, Science 269:1885-1888; Deveroux et al., 1997, Nature 388:300-303). Yeast cells transformed with both a vector encoding a caspase, such as caspase-8β, and a vector encoding the known caspase inhibitor p35 were used to confirm that the cytotoxicity observed in the caspase-8β transformants following expression of the caspase-8β was attributable to caspase-8β activity. Such transformants were also used to demonstrate the operability of this screening method for identifying caspase inhibitors. Furthermore, such transformants may be used to analyze dosage effects of both the caspase and the inhibitor within the transformant cell.

Yeast cells were transformed with both a vector encoding caspase-8β and a vector encoding a known caspase inhibitor as follows. The gene encoding p35 was cloned into a yeast-based expression vector designated YCpGAL1-U (wherein U designates a URA3 selectable marker) to yield a YCpGAL1-p35-U inhibitor vector. Yeast cells were co-transformed with the inhibitor vector, YCpGAL1-p35-U, and with the caspase vector, YCpGAL1-caspase-8β-L, to yield a double transformant. Double transformants were plated on SC-leucine-uracil medium supplemented with dextrose to repress GAL1 promoter function. Serial ten-fold dilutions of individual double transformants grown in SC-leucine-uracil medium containing dextrose were prepared and spotted onto SC-leucine-uracil medium comprising dextrose or galactose. The ability of the p35 caspase inhibitor to prevent caspase-8β-induced cytotoxicity in the double transformant was manifested as the ability of the double transformant to survive incubation on galactose-containing test medium which comprised an inducer of the caspase-8P vector and which did not comprise a repressor of the caspase-8β vector. The ability of the p35 caspase inhibitor to reduce caspase-8β-induced cytotoxicity in the double transformant was manifested as ability of the double transformant to survive incubation on the test medium for a longer period than the period that a yeast cell transformed with the caspase vector, but not with the inhibitor vector, can survive using the same medium. Double transformants which were provided p35 survived on this medium, while double transformants which were not provided p35 did not survive on this medium.

Similarly, yeast cells may be transformed with both a caspase vector, as described herein, and a test vector which encodes a potential caspase inhibitor to yield a double transformant suitable for screening. The ability of the potential caspase inhibitor to prevent caspase-induced cytotoxicity in the double transformant is manifested as ability of the double transformant to survive incubation on or in test medium which comprises an inducer of the caspase vector and which does not comprise a repressor of the caspase vector. The ability of the potential caspase inhibitor to reduce caspase-induced cytotoxicity in the double transformant is manifested as ability of the double transformant to survive incubation on or in the test medium for a longer period than the period that a yeast cell transformed with the caspase vector, but not with the test vector, can survive using the same medium. Individual viable colonies of double transformant are isolated, and the test vector comprising the unknown cDNA is isolated. Subsequent sequencing of the potential caspase inhibitor may identify a gene of known or unknown function, the product of which is capable of inhibiting caspase-induced cytotoxicity.

The ability of a test vector to decrease LacZ expression in a yeast cell transformed with both the test vector and a control vector comprising lacZ operably linked to the GAL1 promoter may be assessed in order to rule out the possibility that the test vector is down-regulating expression from GAL1, rather than inhibiting caspase activity.

The ability of a compound encoded by a test vector to inhibit caspase activity may be further confirmed by comparing a viability indicator of a yeast cell transformed with the test vector and a caspase vector with the same viability indicator of a yeast cell transformed with the test vector, but not with the caspase vector. If the test vector does not affect the viability indicator of this latter cell, then the ability of the compound to inhibit caspase activity is confirmed.

The test vector used in the screening method described in this Example may, for example, be a vector comprising an isolated nucleic acid selected from a multi-copy human cDNA library. For example, a library of multi-copy vectors, each expressing a random human cDNA sequence from the constitutive GPD promoter, may be used. Such vectors have been described (Schild et al., 1990, Proc. Natl. Acad. Sci. USA 87:2916-2920). Alternately, a cDNA library comprising size-selected cDNAs derived from mRNAs obtained from human brain cells and operably linked to the GAL1 promoter in a URA3 2 μm vector may be used in the screening method described herein.

In the screening method described in this Example, double transformant yeast cells were used, wherein the double transformants comprise yeast cells transformed with both a test vector, such as one derived from a human cDNA library linked to the GAL1-promoter in the URA3 2 μm vector, and a caspase-8β vector, such as the YCpGAL1-caspase-8β-L vector. Alternately, single transformant yeast cells may be used, wherein the single transformants comprise yeast cells having a self-activating caspase operably linked to a promoter and integrated into the genome of the cells, and wherein the cells are transformed with a test vector. For example, test vectors may be used to transform yeast cells in which the GAL1-caspase-8β-L construct described herein are integrated into the genome at the LYS2 locus of yeast strain MBY5.

### Example 3

### Identification of Caspase Inhibitors

A screening method has been used to identify numerous products encoded by human cDNA molecules as caspase inhibitors.

The screening method used in this Example was performed as follows. A yeast double transformant was prepared by transforming S. cerevisiae strain FY250 with two yeast expression vectors. The first yeast expression vector comprised cDNA encoding a self-activating form of human caspase-8β operably linked to a GAL1 promoter and a LEU2 selectable marker. This first vector was a single copy vector and was designated YCpGAL1-caspase-8β-L. A plurality of second yeast expression vectors was used, each comprising a single random human cDNA sequence operably linked to a GAL1 promoter and a URA3 selectable marker. The cDNA sequences used to generate the plurality of second yeast expression vectors were obtained from a library of cDNAs prepared from human brain cells. The second vectors were multicopy vectors.

Double transformants were propagated on plates containing SC-leucine-uracil agar comprising dextrose. Colonies of double transformants were replica plated on plates containing medium comprising galactose, whereby expression of caspase-8β and of any product encoded by the cDNA of the second vector was induced. Only a small percentage of double transformants survived on the galactose-containing medium, and these double transformants were recovered as individual colonies. The second vector was recovered from individual colonies of double transformants which were able to survive on the galactose-containing medium and used, along with the first vector, to transform fresh FY250 cells. Subsequent analysis of these freshly double-transformed cells confirmed that the product encoded by the second vector was capable of suppressing caspase-8β-induced cell death. The identity of individual products encoded by the cDNA of individual second vectors was established by determining the nucleotide sequence of the cDNA and comparing the sequence with databases of known human gene sequences, including Genbank, the EST Database, and the Human Genome Database.

The screening method described in this Example has been used to identify each of the following compounds as a caspase inhibitor: human cytochrome b, human tat binding protein (also known as TBP, SUG1, and 26S proteosomal p45 subunit ATPase), human mitochondrial loop attachment site, a glutamate-binding subunit of a human NMDA receptor complex, human myelin basic protein, human synaptophysin p38, human snRNP protein B, human protein 1 (expressed from a mitochondrial gene encoding a product designated 'protein 1'), human ubiquitin C-terminal hydrolase (also known as UHX1), human tissue inhibitor of metalloprotease-3, human MHC HLA-DRw12-MHC class II β chain, human transglutaminase, human death associated protein 1 (also known as DAP-1), and human hnRNP D.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of determining whether a test compound is a caspase modulator, the method comprising
providing a functional eukaryotic caspase to a first yeast cell;
providing said test compound to said first cell; and
comparing a viability indicator of said first cell with the same viability indicator of a second yeast cell which was provided said caspase and which was not provided said test compound, wherein a difference between said viability indicator of said first cell and said viability indicator of said second cell is an indication that the compound is a caspase modulator.

2. The method of claim 1, wherein said first cell and said second cell are of the same species selected from the group consisting of a Saccharomyces cerevisiae cell and a Schizosaccharomyces pombe cell.

3. The method of claim 2, wherein said Saccharomyces cerevisiae is selected from the group consisting of strain FY250 and strain MBY5.

4. The method of claim 1, wherein said caspase is provided to the interior of said first cell by providing a caspase vector to said first cell, said caspase vector comprising an isolated nucleic acid encoding a self-activating caspase and a promoter operably linked to said isolated nucleic acid.

5. The method of claim 4, wherein said caspase vector further comprises a second isolated nucleic acid encoding a self-activating form of a second eukaryotic caspase.

6. The method of claim 5, wherein both of said isolated nucleic acids are operably linked to said promoter.

7. The method of claim 4, wherein said promoter is selected from the group consisting of an inducible promoter, a repressible promoter, and a promoter which is both inducible and repressible.

8. The method of claim 7, wherein said promoter is an inducible promoter, and wherein said first cell is incubated first in the absence of an inducer of said promoter and thereafter in the presence of said inducer.

9. The method of claim 7, wherein said promoter is a GAL1 promoter.

10. The method of claim 1, wherein said caspase is provided to said first cell by providing an expressible vector encoding said caspase to said first cell and thereafter expressing said caspase.

11. The method of claim 1, wherein said caspase is provided to said first cell by integrating an isolated nucleic acid encoding said caspase into the genome of said first cell and thereafter expressing said caspase.

12. The method of claim 1, wherein said caspase is selected from the group consisting of human caspase 1, human caspase 2, human caspase 3, human caspase 4, human caspase 5, human caspase 6, human caspase 7, human caspase 8, human caspase 9, human caspase 10, and granzyme B.

13. The method of claim 1, wherein said viability indicator is selected from the group consisting of cell number, cell refractility, cell fragility, cell size, number of cellular vacuoles, a stain which distinguishes live cells from dead cells, methylene blue stain, bud size, bud location, nuclear morphology, and nuclear staining.

14. An anti-caspase gene vector comprising an isolated nucleic acid which encodes a caspase inhibitor selected from the group consisting of human cytochrome b, human tat binding protein, human mitochondrial loop attachment site, a glutamate-binding subunit of a human NMDA receptor complex, human myelin basic protein, human synaptophysin p38, human snRNP protein B, human protein 1, human ubiquitin C-terminal hydrolase, human tissue inhibitor of metalloprotease-3, human MHC HLA-DRw12-MHC class II β chain, human transglutaminase, human death associated protein 1, and human hnRNP D.

15. A method of modulating apoptosis in a eukaryotic cell, the method comprising providing a caspase modulator to the interior of said cell, wherein said caspase modulator is selected from the group consisting of human cytochrome b, human tat binding protein, human mitochondrial loop attachment site, a glutamate-binding subunit of a human NMDA receptor complex, human myelin basic protein, human synaptophysin p3 8, human snRNP protein B, human protein 1, human ubiquitin C-terminal hydrolase, human tissue inhibitor of metalloprotease-3, human MHC HLA-DRw12-MHC class II β chain, human transglutaminase, human death associated protein 1, and human hnRNP D.

16. The method of claim 15, wherein said caspase modulator is a caspase inhibitor, and wherein said cell is an affected cell in a human patient afflicted with a misapoptotic disease.

17. The method of claim 15, wherein said caspase modulator is a caspase activator, and wherein said cell is an affected cell in a human patient afflicted with a misapoptotic disease.

18. A yeast cell comprising an isolated nucleic acid which encodes a caspase, said isolated nucleic acid being operably linked to a promoter.

19. A gene vector comprising an isolated nucleic acid encoding a caspase, said isolated nucleic acid being operably linked to an inducible promoter.

20. The gene vector of claim 19, further comprising a second isolated nucleic acid encoding a second caspase, wherein said second isolated nucleic acid is also operably linked to said promoter.
